(19) United States
(12) Patent Application Publication
(10) Pub. No.: US 2024/0150398 A1
(43) Pub. Date: May 9, 2024

(54) PEPTIDES AND CONJUGATES THEREOF AS ACE-2 AND S1 SUBUNIT MIMICS AGAINST SEVERE ACUTE RESPIRATORY SYNDROME CORONAVIRUS-2 (SARS-COV2) INFECTION

(71) Applicant: NEUOME PEPTIDES PTE. LTD., Singapore (SG)

(72) Inventor: Rajanikanth Vangala, Bangalore (IN)

(21) Appl. No.: 18/141,298

(22) Filed: Apr. 28, 2023

Related U.S. Application Data
(63) Continuation of application No. PCT/IN2021/051030, filed on Oct. 28, 2021.

(30) Foreign Application Priority Data
Oct. 28, 2020 (IN) 202041042101

(51) Int. Cl. C07K 7/06; A61K 47/69; A61P 31/14; C07K 7/08

(52) U.S. Cl. CPC C07K 7/06; A61K 47/6929; A61P 31/14; C07K 7/08

(57) ABSTRACT

The present invention provides peptides and conjugates thereof, as ACE-2 and S1 subunit mimicking peptides for the prevention and control of Severe Acute Respiratory Syndrome Coronavirus-2 (SARS-CoV2) infection by preventing the binding of Severe Acute Respiratory Syndrome Coronavirus-2 to the target cells.

### RELATED APPLICATIONS

This application claims priority under 35 U.S.C. § 120 to, and is a continuation of, co-pending International Application PCT/IN2021/051030, filed Oct. 28, 2021 and designating the US, which claims priority to IN Application 202041042101, filed Oct. 28, 2020, such IN Application also being claimed priority to under 35 U.S.C. § 119. These IN and International applications are incorporated by reference herein in their entireties.

### FIELD

The present invention relates to the fields of proteomics and virology, more particularly to peptides and conjugates thereof, as ACE-2 and S1 subunit mimicking peptides for the prevention and control of Severe Acute Respiratory Syndrome Coronavirus-2 (SARS-CoV2) infection by preventing the binding of Severe Acute Respiratory Syndrome Coronavirus-2 (hereinafter called as “SARS-CoV2”) to the target cells.

### BACKGROUND

Background description includes information that may be useful in understanding the present invention. It is not an admission that any of the information provided herein is prior art or relevant to the presently claimed invention, or that any publication specifically or implicitly referenced is prior art.

Severe Acute Respiratory Syndrome (SARS) is a highly contagious and potentially lethal viral disease characterized by pronounced respiratory symptoms and pneumonia. Severe Acute Respiratory Syndrome Coronavirus-2 (SARS CoV-2) has emerged as a global pandemic and there is a need for the development of biomolecules, vaccines, drugs which will help in prophylactic/preventive as well as therapeutic applications against SARS CoV-2. The rapid emergence and spread of this disease have led to intense efforts to develop effective methods of prevention and treatment. These efforts have led to the isolation of a unique coronavirus that causes SARS and to the complete sequencing of this virus's genome. The genome of the SARS virus has been completely sequenced and based on the sequence information and on a knowledge of the life cycle of other coronaviruses, it is believed that entry into host cells by the SARS virus is mediated by a surface protein, designated as “S,” which is analogous to the HIV-1 envelope glycoprotein. Some coronaviruses cleave the S protein into two fragments called S1 (which mediates receptor binding) and S2 (which mediates fusion of the virus to the host cell membrane). In other coronaviruses, including the one causing SARS, the S protein is not cleaved. Nevertheless, the S1 and S2 domains can be identified and initial receptor binding is apparently still mediated by S1.

The virus causing SARS (SARS-CoV) does not belong to any of the previously defined genetic and serological coronavirus groups and the SARS-CoV S protein that mediates virus entry into receptor-bearing cells is also distinct from those of other coronaviruses (Marra, et al., Science 300: 1399-1404 (2003). Due the distinct nature of the S proteins, SARS-CoV does not utilize any previously identified coronavirus receptors to infect cells. Rather, angiotensin-converting enzyme 2 (ACE2) serves as a functional receptor for this virus.

Present antiviral peptides for SARS are designed based on the genomic sequence of SARS virus and are not targeted towards a specific interaction or the function. As discussed above, ACE-2 is a potent target which plays a major role in viral interaction to cell surface and also renin-angiotensin system. This regulatory aspect of ACE-2 plays an important role in eliciting inflammatory lung disease in addition to its well-known role in regulation of blood pressure and balance of electrolytes. Another protease TMPRSS2 is produced by host cells where SARS virus attach. TMPRSS2 processes the S protein proteolytically to prime it to bind to ACE-2 protein. Several TMPRSS2 inhibitors are in clinical trials. To date, no effective vaccines or therapies are available against the severe acute respiratory syndrome coronavirus 2 (SARS-CoV-2), the causative pandemic agent of the coronavirus disease 2019 (COVID-19). Due to their safety, efficacy and specificity, peptide inhibitors hold great promise for the treatment of newly emerging viral pathogens. Based on the known structures of viral proteins and their cellular targets, antiviral peptides can be rationally designed and optimized. The resulting peptides may be highly specific for their respective targets and particular viral pathogens or exert broad antiviral activity. There are studies on peptides inhibiting SARS-CoV-2 entry and various strategies have been proposed to design peptides targeting the ACE2 receptor or the viral spike protein and its activating proteases furin, transmembrane serine protease 2 (TMPRSS2), or cathepsin L. However, most of these peptides are new chemical entities and virus may soon develop resistance due to selective pressure. Thus, there is a need to develop novel and effective peptide-based drugs that focus on the interaction between ACE-2 and Spike proteins S1 or S2.

Presently, definitive and confirmative therapies, control or preventive agents for coronavirus infection are lacking. Though the genome sequence of SARS-CoV has been determined, however, much remains to be learnt about this virus, and means and methods for diagnostics and treatment of the virus and the syndrome are needed.

### SUMMARY

In one aspect, the present invention provides a mimic peptide for specifically binding S1 spike protein of SARS CoV2 virus comprising an amino acid sequence of SEQ ID NO: 3 or SEQ ID NO: 4. In an aspect, the present invention provides a mimic peptide complex comprising the amino acid sequence of SEQ ID NO: 3 and SEQ ID NO: 4.

In an aspect, the present invention provides a conjugate for specifically binding S1 spike protein of SARS CoV2 virus comprising a mimic peptide having an amino acid sequence of SEQ ID NO: 3 conjugated with 10 nm to 20 nm nanoparticles.

In an aspect, the present invention provides a conjugate for specifically binding S1 spike protein of SARS CoV2 virus comprising a mimic peptide having an amino acid sequence of SEQ ID NO: 4 conjugated with 10 nm to 20 nm nanoparticles.

In an aspect, the present invention provides a conjugate for specifically binding S1 spike protein of SARS CoV2 virus comprising a mimic peptide complex having an amino acid sequence of SEQ ID NO: 3 and SEQ ID NO: 4 conjugated with 10 nm to 20 nm nanoparticles.

In another aspect, the present invention provides a pharmaceutical composition comprising a mimic peptide of SEQ ID NO: 3 or SEQ ID NO: 4 or a peptide complex comprising SEQ ID NO: 3 and SEQ ID NO: 4, and a pharmaceutically acceptable carrier. In an embodiment, the pharmaceutical composition comprises a conjugate comprising the mimic peptide of SEQ ID NO: 3, SEQ ID NO: 4 or the peptide complex comprising SEQ ID NO: 3 and SEQ ID NO: 4 conjugated with 10 nm to 20 nm nanoparticles. In an embodiment, the pharmaceutically acceptable carriers comprise water, saline, phosphate buffered saline, dextrose, glycerol, ethanol, isotonic agents, selected from sugars, polyalcohols such as mannitol, sorbitol, or combination thereof.

In an aspect, the present invention provides a method of inhibiting the SARS CoV2 infection in a subject comprising administering to the subject an effective amount of the mimic peptide of SEQ ID NO: 3 or SEQ ID NO: 4 or the peptide complex comprising SEQ ID NO: 3 and SEQ ID NO: 4 thereby inhibiting or blocking the interaction of SARS-CoV2 spike protein with ACE-2 receptor of a host cell of the subject. In an embodiment, the method of inhibiting the SARS CoV2 infection in a subject wherein comprises administering to the subject an effective amount of the conjugate comprising the mimic peptide of SEQ ID NO: 3 or SEQ ID NO: 4 or the peptide complex comprising SEQ ID NO: 3 and SEQ ID NO: 4, thereby inhibiting or blocking interaction of SARS-CoV2 spike protein with ACE-2 receptor of a host cell of the subject.

In an aspect, the present invention provides a method of treating or effecting prophylaxis of SARS CoV2 infection in a subject comprising administering to the subject an effective amount of the mimic peptide of SEQ ID NO: 3 or SEQ ID NO: 4 or the peptide complex comprising SEQ ID NO: 3 and SEQ ID NO: 4, thereby inhibiting or blocking interaction of SARS-CoV2 spike protein with ACE-2 receptor of a host cell in the subject. In an embodiment, the method of treating or effecting prophylaxis of SARS CoV2 infection in a subject, wherein comprises administering to the subject an effective amount of the conjugate the mimic peptide of SEQ ID NO: 3 or SEQ ID NO: 4 or the peptide complex comprising SEQ ID NO: 3 and SEQ ID NO: 4, thereby inhibiting the interaction of SARS-CoV2 spike protein with ACE-2 receptor of a host cell in the subject.

In an aspect, the present invention provides use of mimic peptide comprising an amino acid sequence of SEQ ID NO: 3 or SEQ ID NO: 4 or the peptide complex comprising SEQ ID NO: 3 and SEQ ID NO: 4 for inhibiting or blocking interaction of SARS-CoV2 spike protein with ACE-2 receptor of a host cell in a subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Reference will be made to embodiments of the invention, examples of which may be illustrated in the accompanying figures. These figures are intended to be illustrative, not limiting. Although the invention is generally described in the context of these embodiments, it is understood that it is not intended to limit the scope of the invention to these particular embodiments:

FIG. 1 illustrates interaction of the peptides of SEQ ID NO: 1 to SEQ ID NO: 4 of the present invention, and the virus which binds to ACE-2 receptor on cell surface according to an embodiment of the present invention;

FIGS. 2A-2F illustrates 3D model interaction of peptides of SEQ ID NO: 1 to SEQ ID NO: 6 of the present invention with spike S1 protein depicting 10 different binding possibilities surface according to an embodiment of the present invention;

FIG. 3A illustrates the interaction between ACE-2 and spike S1 protein simulated using the structural information available from the crystal structures of SARS-COV spike according to an embodiment of the present invention;

FIG. 3B illustrates 3D model interaction of peptide 3 of SEQ ID NO: 3 of the present invention to the receptor binding domain (RBD) and other internal region of S1 protein blocking from interacting with ACE2 and further replication of virus according to an embodiment of the present invention;

FIG. 3C illustrates 3D model interaction of peptide 4 of SEQ ID NO: 4 of the present invention to the receptor binding domain (RBD) and other internal region of S1 protein blocking from interacting with ACE2 and further replication of virus according to an embodiment of the present invention;

FIG. 4A illustrates interaction of the peptide 3 of SEQ ID NO: 3 of the present invention, and the virus which binds to ACE-2 receptor on cell surface according to an embodiment of the present invention;

FIG. 4B illustrates interaction of the peptide 4 of SEQ ID NO: 4 of the present invention, and the virus which binds to ACE-2 receptor on cell surface according to an embodiment of the present invention;

FIG. 4C illustrates interaction of the peptide complex of SEQ ID NO: 3 and SEQ ID NO: 4 of the present invention, and the virus which binds to ACE-2 receptor on cell surface according to an embodiment of the present invention;

FIGS. 5A-5B depict detection of SARS-CoV2 Spike S1 protein with Peptide 3 (P3) of SEQ ID NO: 3 of the present invention conjugated with gold nanoparticles;

FIGS. 6A-6B depict detection of SARS-CoV2 Spike S1 protein with Peptide 4 (P4) of SEQ ID NO: 4 of the present invention conjugated with gold nanoparticles;

FIG. 7A depicts an experiment with ACE-2 expressing cells HEK293 cells showing protein-protein interactions of His-Tag recombinant S1 protein and recombinant ACE-2 for binding with and without Peptide-3 of SEQ ID NO: 3 of the present invention;

FIGS. 7B-D depict an experiment with ACE-2 expressing cells HEK293 cells showing protein-protein interactions of His-Tag recombinant S1 protein and recombinant ACE-2 for binding with and without Peptide-4 of SEQ ID NO: 4 of the present invention; and

FIG. 8 depicts results of the ACE-2 receptor binding to spike S1 protein with combination of peptide 3 of SEQ ID NO: 3 and peptide 4 of SEQ ID NO: 4 as a peptide complex at Ing concentration each.

### DETAILED DESCRIPTION

The problems and shortcomings associated with the prior art, techniques and approaches are overcome by the present invention as described below in the preferred embodiments.

The present invention provides novel peptides, conjugates and compositions for antiviral therapy against enveloped virus such as for example SARS-COV-2. The present invention also provides the use of the peptides individually or in combination with or without modifications or conjugations as antiviral agents.

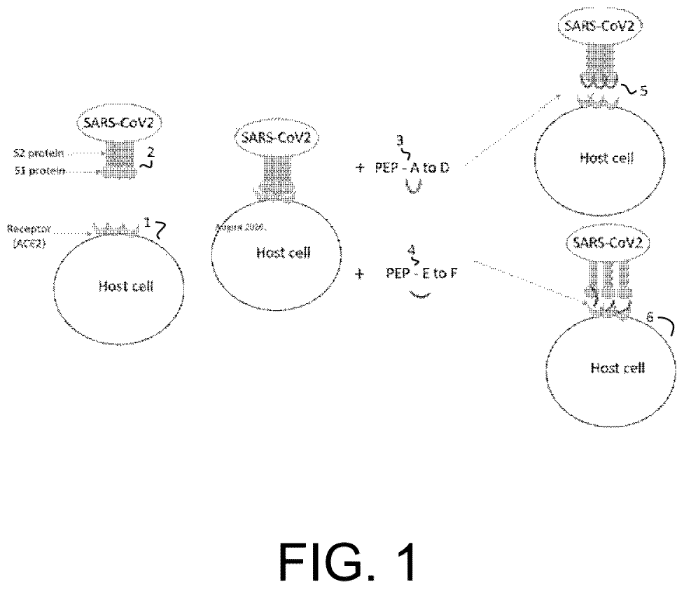

In an aspect, the present invention provides novel peptides that inhibit the binding of virus to the host cell by mimicking ACE-2 receptor. The mimic peptides can have an amino acid sequence of SEQ ID NO: 1 (YQKDHQM), SEQ ID NO: 2 (QKHYQMK), SEQ ID NO: 3 (QTFDKNHEDLYLQAKQLK), or SEQ ID NO: 4 (DKFNHEAEDLFY), individually or in combination as a peptide complex. The said peptides can individually or in combination with other peptides as peptide conjugates or as modified peptides or peptide complex and can bind to SARS-CoV2 spike S1 protein subunit and competitively inhibit the binding of SARS-CoV2 spike protein S1 subunit to the host cell mimicking ACE-2 receptor. FIG. 1 shows that the said peptides designated as Peptides A to D (SEQ ID NO: 1 to SEQ ID NO: 4) respectively can mimic the ACE-2 receptor and prevent the binding of virus to the said ACE-2 receptor thereby preventing the viral infection of the host cell.

In another aspect, the present invention provides peptides that have affinity to bind to SARS-CoV2 spike protein S1 subunit. The said peptides of SEQ ID NO: 5 and/or SEQ ID NO: 6 can have affinity to bind to SARS-CoV2 spike protein S1 subunit. The said peptides can have an amino acid sequence of QTNKYQFQTNKYQF or KYQFQTNKYQFQTN respectively. The said peptides can individually or in combination with other peptides conjugate or modify and can bind to SARS-CoV2 spike S1 protein subunit and competitively inhibit the binding of SARS-CoV2 spike protein S1 subunit to the host cell. The peptides are designated as Peptide E (PepE-SEQ ID NO: 5) to F (PepF-SEQ ID NO: 6) and as (label 4) in FIG. 1. Peptides E and F (SEQ ID NO: 5 and SEQ ID NO: 6) individually or in combinations with other peptides conjugated or modified, mimic the SARS CoV-2 spike protein S1I subunit and bind to S1 proteins (2) while trimerization of S1 protein and also bind to ACE-2 receptor (1) thus competing away the virus from binding and entering the cells for infection. The binding of the peptides of SEQ ID NO: 5 (PEP-E) and SEQ ID NO: 6 (PEP-F) of the present invention to the said S1 protein subunit thus can stop or prevent the virus entering into the cell and hence can prevent and/or stop infection of the host cell.

FIG. 1 illustrates interaction of the peptides of SEQ ID NO: 1, SEQ ID NO: 2, SEQ ID NO: 3 and SEQ ID NO: 4 and SEQ ID NO: 5 and SEQ ID NO: 6 of the present invention, and the virus which binds to spike protein or ACE-2 receptor on cell surface respectively according to an embodiment of the present invention. FIG. 1 illustrates that the mimic peptides (label 3,4) can alone or in combination can mimic the ACE-2 receptor and prevent the binding of virus (label 2) to the said ACE-2 receptor (label 1) thereby preventing the viral infection of the host cell (label 6). The ACE-2 inhibitor peptides of SEQ ID NO: 1 to SEQ ID NO: 4 are designated as peptide A (PepA), peptide B (PepB), peptide C (PepC) and peptide D (PepD) in said FIG. 1. The host cells (label 6) which express Angiotensin Converting Enzyme Receptor-2 (ACE-2) (label 1) usually bind the receptor binding domain (RBD) of spike S1 protein in SARS-CoV2 virus (label 2). Based on this interaction, a specific approach of identifying the interacting amino acids was devised to develop the peptides of the present invention, which can interact with receptor binding domain (RBD) of spike S1 protein in SARS-CoV2 virus (label 2). The interaction between ACE-2 (1) and spike S1 protein was simulated using the structural information available from the crystal structures of SARS-COV spike (Protein data Bank-PDB code 6ACK) and ACE-2 (PDB code 1R42). The structures were analyzed and interaction interfaces were identified using DOCK software (http://zdock.umassmed.edu/). The interacting amino acids between spike S1 protein and ACE-2 were identified. The interacting amino acids were found to be not just linear but were several amino acids were interacting with more than one amino acid suggesting that their interactions were more important. Using such amino acids and others, several combinations of amino acids resulting in peptides SEQ ID NO: 1, SEQ ID NO: 2, SEQ ID NO: 3, and SEQ ID NO: 4 of the present invention were generated.

In a further aspect of the invention, peptides in accordance with an embodiment of the present invention may be coupled/linked or conjugated to each other. The peptides may be linear and/or looped/cyclic. A combination peptide or a conjugate peptide may also constitute of more than two peptides. The peptides of the invention can be linked directly or indirectly via for instance a spacer of variable length. Furthermore, the peptides can be linked covalently or non-covalently. They may also be part of a fusion protein or conjugate.

In an embodiment, the present invention provides conjugates or modified peptides where the peptides of SEQ ID NO: 1 to SEQ ID NO: 6 of the present invention can further be conjugated or modified or formulated to specific protecting compounds from proteolysis and degradation. The protecting compound can be polyethylene glycol. The conjugated or modified peptides or formulations can be used for treatment or prevention of SARS CoV-2. The peptides can be used individually or in combinations to achieve protection against viral infection or for therapy.

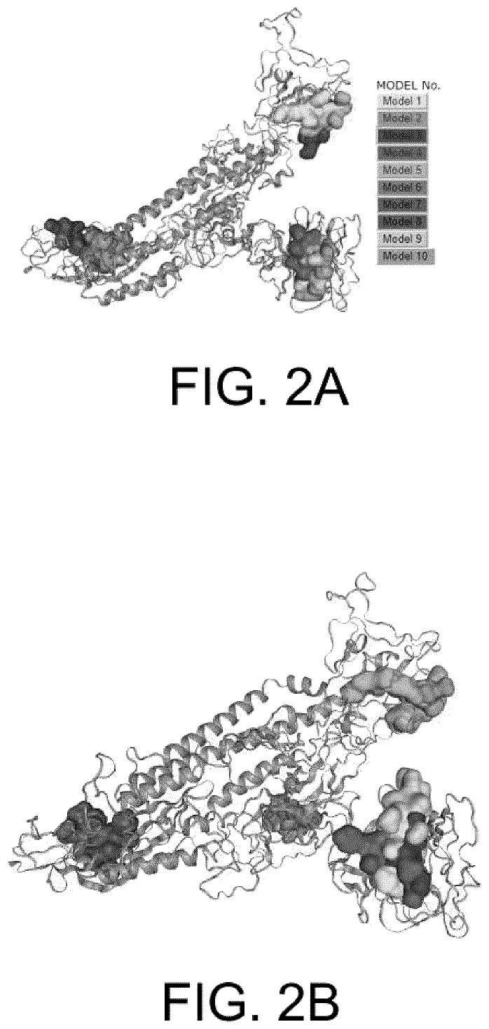

Out of the several peptides generated by combinations of ACE-2 amino acids interacting with spike S1 protein from table 1 and FIGS. 2A to 2F, their binding strengths were calculated using docking score.

The docking scores of the peptides are shown in Table 1 below:

TABLE 1

| PEPTIDE | SEQ ID NO: | DOCKING SCORE |
|---|---|---|
| PEPTIDE 1 (PEP-A) | SEQ ID NO: 1 | −176.931 |
| PEPTIDE 2 (PEP-B) | SEQ ID NO: 2 | −178.854 |
| PEPTIDE 3 (PEP-C) | SEQ ID NO: 3 | −203.819 |
| PEPTIDE 4 (PEP-D) | SEQ ID NO: 4 | −230.184 |
| PEPTIDE 5 (PEP-E) | SEQ ID NO: 5 | −213.3 |
| PEPTIDE 6 (PEP-F) | SEQ ID NO: 6 | −224.1 |

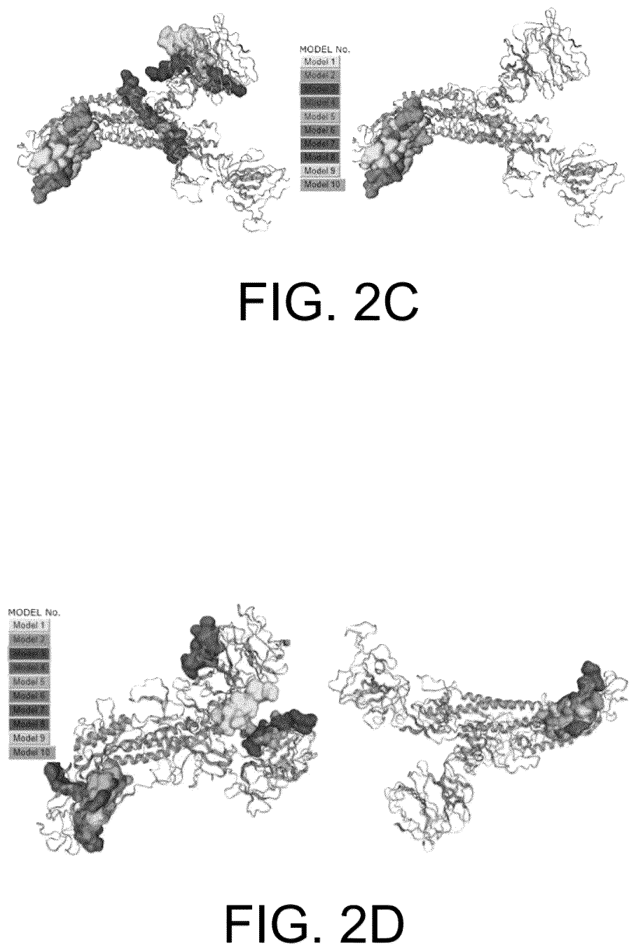

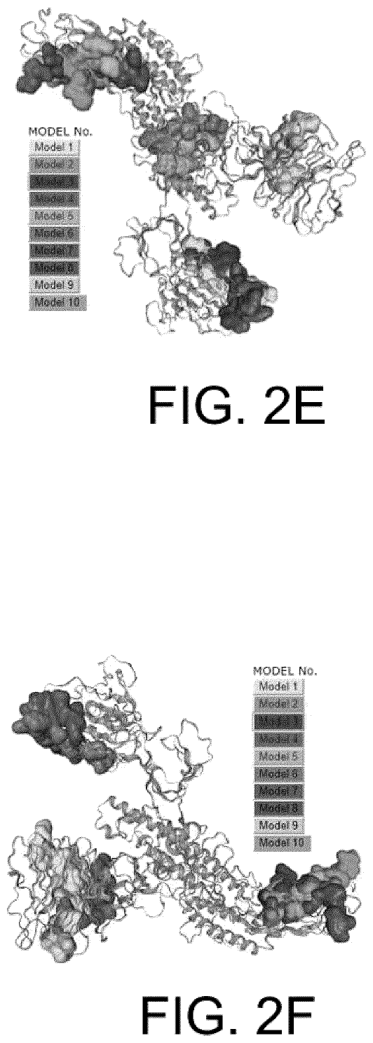

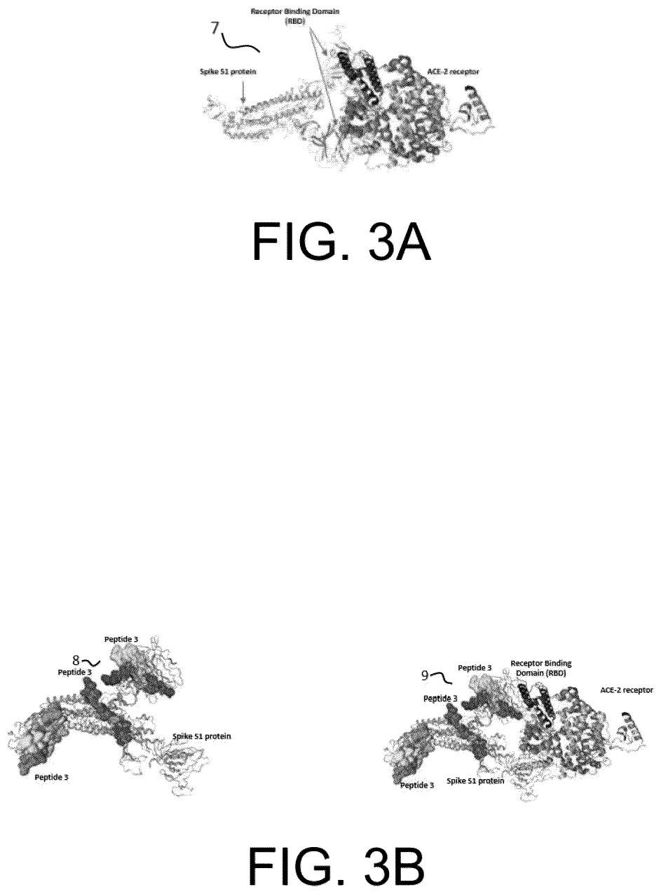

As illustrated in FIGS. 3A-3C, the interaction of peptide 3 of SEQ ID NO: 3 and peptide 4 of SEQ ID NO: 4 of the present invention were found to be interacting with spike S1 protein and showed that there are 10 different binding possibilities with different docking scores. Based on the best docking score, the peptide 3 of SEQ ID NO: 3 (FIG. 3B) and peptide 4 of SEQ ID NO: 4 (FIG. 3B) of the present invention were selected for further evaluation.

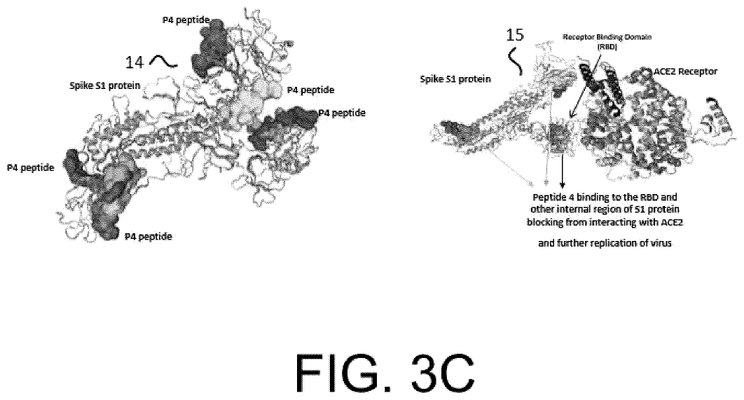

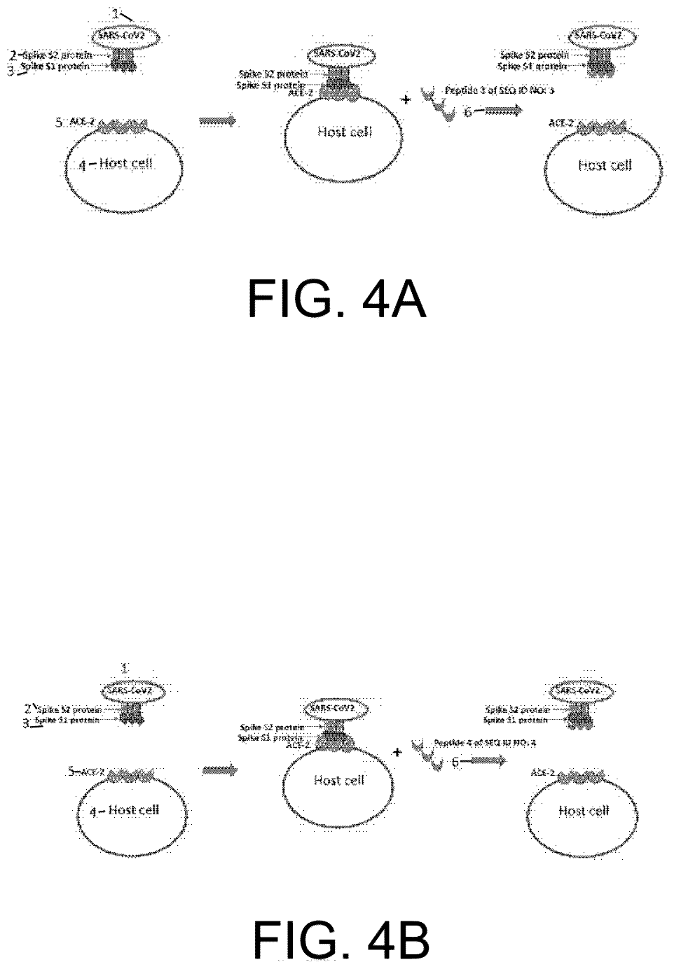

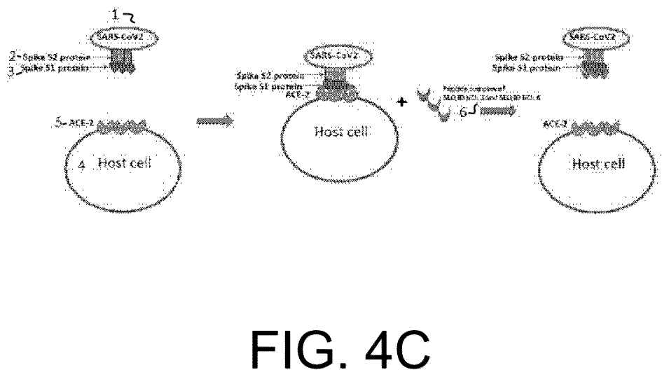

Thus, in an aspect, the present invention provides a mimic peptide for specifically binding S1 spike protein of SARS CoV2 virus comprising an amino acid sequence of SEQ ID NO: 3 (QTFDKNHEDLYLQAKQLK) or SEQ ID NO: 4 (DKFNHEAEDLFY). The mimic peptide can be conjugated with protecting compounds such as polyethylene glycol protecting the peptides from proteolysis and/or degradation. The said peptide can individually or in combination with other peptides conjugate or modify and can bind to SARS-CoV2 spike S1 protein subunit and competitively inhibit the binding of SARS-CoV2 spike protein S1 subunit to the host cell. SEQ ID NO: 3 or SEQ ID NO: 4 of the present invention interacts with the virus which binds to ACE-2 receptor on cell surface according to an embodiment of the present invention shown in FIG. 4A-4B. In an embodiment, the peptide complex of SEQ ID NO: 3 and SEQ ID NO: 4 of the present invention interact with the virus which binds to ACE-2 receptor on cell surface as shown in FIG. 4C.

In an embodiment, the present invention provides a mimic peptide for specifically binding S1 spike protein of SARS CoV2 virus comprising an SEQ ID NO: 4 (DKFNHEAEDLFY). The mimic peptide can be conjugated with protecting compounds to protect the mimic peptides from proteolysis and/or degradation. The protecting compounds can be polyethylene glycol. The said peptide conjugate can in combination with other peptides conjugate or modify and can bind to SARS-CoV2 spike S1 protein subunit and competitively inhibit the binding of SARS-CoV2 spike protein S1 subunit to the host cell.

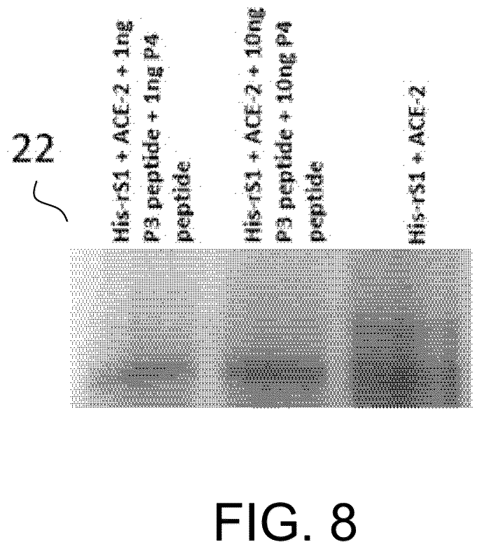

In an embodiment, the present invention provides a mimic peptide complex for specifically binding S1 spike protein of SARS CoV2 virus. The peptide complex comprises the amino acid sequence of SEQ ID NO: 3 (QTFDKNHEDLYLQAKQLK) and SEQ ID NO: 4 (DKFNHEAEDLFY). The said peptide complex can bind to SARS-CoV2 spike S1 protein subunit and competitively inhibit the binding of SARS-CoV2 spike protein S1 subunit to the host cell. FIG. 8 depicts that in presence of combination i.e. peptide complex of peptide 3 of SEQ ID NO: 3 and peptide 4 of SEQ ID NO: 4, the ACE-2 receptor binding to spike S1 protein. It has been observed that peptide 3 of SEQ ID NO: 3 and peptide 4 of SEQ ID NO: 4 individually inhibit or block the interaction of SARS-CoV2 spike protein with ACE-2 receptor of a host cell at a concentration of lOng. It has been surprisingly found that the peptide complex comprising peptide 3 of SEQ ID NO: 3 and SEQ ID NO: 4 in combination can completely inhibit or block the interaction of SARS-CoV2 spike protein with ACE-2 receptor of a host cell at even at a concentration of 1 ng.

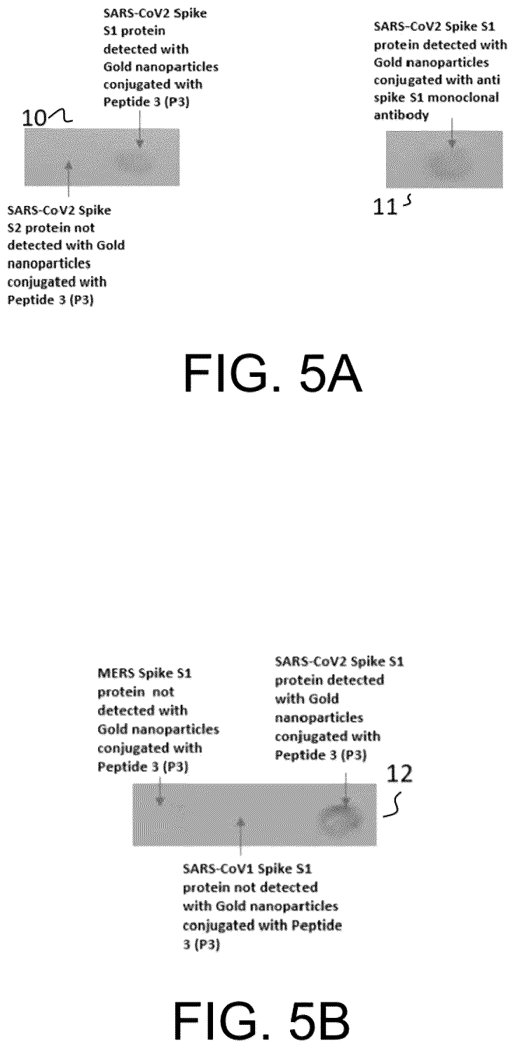

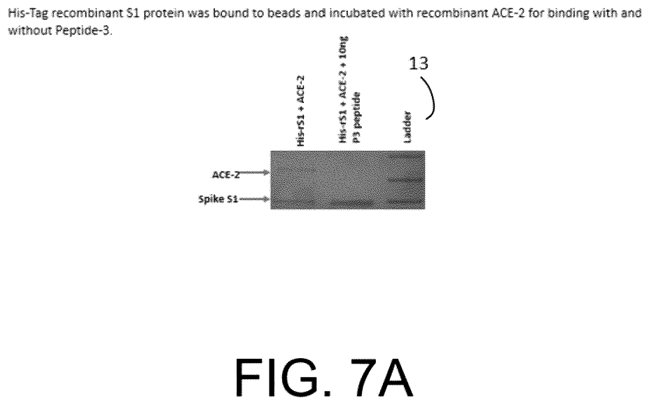

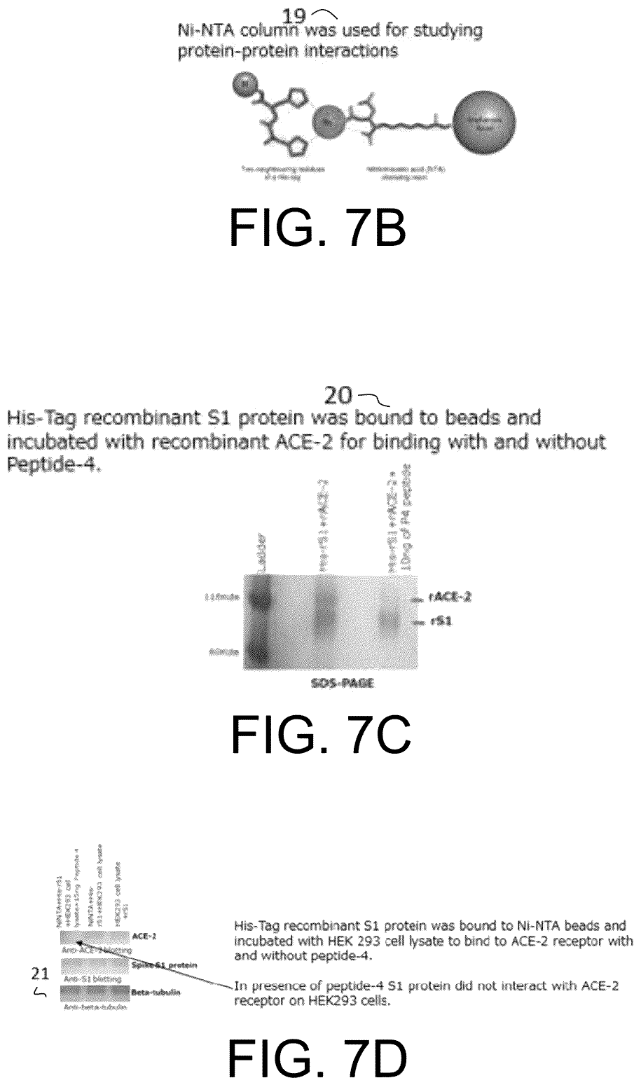

In another aspect, the present invention provides a conjugate for specifically binding S1 spike protein of SARS CoV2 virus comprising a mimic peptide having an amino acid sequence of SEQ ID NO: 3 conjugated with 10 nm to 20 nm nanoparticles. The mimic peptide conjugate of the present invention can detect the spike S1 protein specifically. FIG. 5 A shows that the mimic peptide conjugate having the mimic peptide SEQ ID NO: 3 (P3) detects the S1 protein (label 10 and 11). FIG. 5B shows that the mimic peptides are specific to S1 protein and cannot bind the spike proteins of MERS and SARS-CoV1 viruses (label 12). The mimic peptide of SEQ ID NO: 3 (P3) or its conjugate blocks the interaction between ACE-2 receptor and SARS-CoV2 spike S1 protein. The results as depicted in FIG. 7 A at label 13 shows that the interaction between ACE-2 receptor and spike S1 protein of SARS-CoV2 is blocked in presence of peptide SEQ ID NO: 3 (P3).

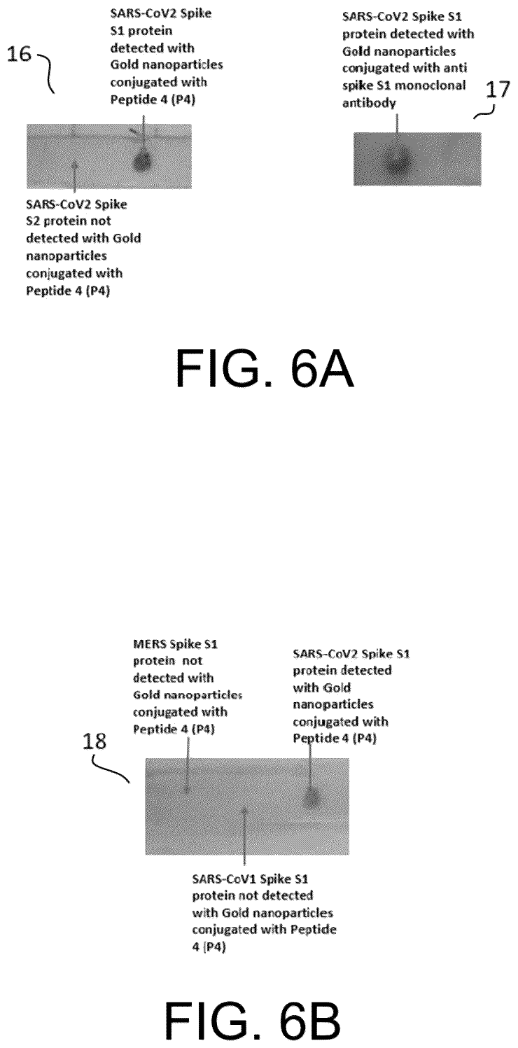

In another aspect, the present invention provides a conjugate for specifically binding S1 spike protein of SARS CoV2 virus comprising a mimic peptide having an amino acid sequence of SEQ ID NO: 4 conjugated with 10 nm to 20 nm nanoparticles. The mimic peptide conjugate of the present invention can detect the spike S1 protein specifically. FIG. 6A shows that the mimic peptide conjugate having the mimic peptide SEQ ID NO: 4 (P4) detects the S1 protein (label 16 and 17). FIG. 6B shows that the mimic peptides are specific to S1 protein and cannot bind the spike proteins of MERS and SARS-CoVl viruses (label 18). The mimic peptide of SEQ ID NO: 4 (P4) or its conjugate blocks the interaction between ACE-2 receptor and SARS-CoV2 spike S1 protein. The results as depicted in FIG. 7B at label 21 shows that the interaction between ACE-2 receptor and spike S1 protein of SARS-CoV2 is blocked in presence of peptide SEQ ID NO: 4 (P4).

In another aspect, the present invention provides a conjugate for specifically binding S1 spike protein of SARS CoV2 virus comprising a mimic peptide complex having an amino acid sequence of SEQ ID NO: 3 and SEQ ID NO: 4 conjugated with 10 nm to 20 nm nanoparticles. The mimic peptide conjugate of the present invention can detect the spike S1 protein specifically.

In an embodiment, the present invention provides a mimic peptide or a peptide complex or a conjugate for specifically binding S1 spike protein of SARS CoV2 virus comprising a mimic peptide having an amino acid sequence of SEQ ID NO: 3, preferably conjugated with 10 nm or 15 nm or to 20 nm nanoparticles.

In another aspect, the present invention provides a pharmaceutical composition comprising the mimic peptide of SEQ ID NO: 3 or SEQ ID NO: 4 or the peptide complex of SEQ ID NO: 3 and SEQ ID NO: 4, and a pharmaceutically acceptable carrier. In an embodiment, the pharmaceutical composition comprises the mimic peptide of SEQ ID NO: 3 or SEQ ID NO: 4 conjugated with 10 nm to 20 nm nanoparticles, preferably conjugated with 10 nm, 15 nm or to 20 nm nanoparticles.

In an aspect, the present invention provides a pharmaceutical composition comprising the mimic peptide complex consisting of SEQ ID NO: 3 and SEQ ID NO: 4 and a pharmaceutically acceptable carrier. In an embodiment, the pharmaceutical composition comprises the mimic peptide complex consisting of SEQ ID NO: 3 and SEQ ID NO: 4 conjugated with 10 nm to 20 nm nanoparticles, preferably conjugated with 10 nm, 15 nm, or to 20 nm nanoparticles.

In an embodiment, the pharmaceutically acceptable carriers comprise water, saline, phosphate buffered saline, dextrose, glycerol, ethanol, isotonic agents, selected from sugars, polyalcohols such as mannitol, sorbitol, or combination thereof.

In an embodiment, the composition of the present invention can be prepared by conventional methods of preparing such peptide formulations and can include pharmaceutical excipients including carriers, and stabilizers. In an embodiment, the composition can be formulated into an oral composition, topical, sub-cutaneous, intravenous, intra-articular, nebulizer or a nasal composition. Preferably, the composition comprising the peptides of the present invention is a nasal spray composition. The composition can preferably be a nebulizer composition.

In an aspect, the present invention provides a method of inhibiting the SARS CoV2 infection in a subject comprising administering to the subject an effective amount of the mimic peptide of SEQ ID NO: 4 thereby inhibiting or blocking the interaction of SARS-CoV2 spike protein with ACE-2 receptor of a host cell of the subject. In an embodiment, the method of inhibiting the SARS CoV2 infection in a subject wherein comprises administering to the subject an effective amount of the conjugate comprising the mimic peptide of SEQ ID NO: 3 or SEQ ID NO: 4 or the peptide complex comprising SEQ ID NO: 3 and SEQ ID NO: 4, thereby inhibiting or blocking interaction of SARS-CoV2 spike protein with ACE-2 receptor of a host cell of the subject.

In an aspect, the present invention provides a method of treating or effecting prophylaxis of SARS CoV2 infection in a subject comprising administering to the subject an effective amount of the mimic peptide of SEQ ID NO: 3 or SEQ ID NO: 4 or the peptide complex comprising SEQ ID NO: 3 and SEQ ID NO: 4, thereby inhibiting or blocking interaction of SARS-CoV2 spike protein with ACE-2 receptor of a host cell in the subject. In an embodiment, the method of treating or effecting prophylaxis of SARS CoV2 infection in a subject, wherein comprises administering to the subject an effective amount of the conjugate comprising the mimic peptide of SEQ ID NO: 3 or SEQ ID NO: 4 or the peptide complex comprising SEQ ID NO: 3 and SEQ ID NO: 4, thereby inhibiting the interaction of SARS-CoV2 spike protein with ACE-2 receptor of a host cell in the subject.

In an aspect, the present invention provides use of mimic peptide comprising an amino acid sequence of SEQ ID NO: 3 or SEQ ID NO: 4, or the peptide complex comprising SEQ ID NO: 3 and SEQ ID NO: 4 for inhibiting or blocking interaction of SARS-CoV2 spike protein with ACE-2 receptor of a host cell in a subject.

The present invention in yet another aspect provides a method of competitively inhibiting the interaction of SARS-CoV2 spike protein with a host cell by providing a peptide of SEQ ID No: 5 or SEQ ID NO: 6 having an affinity to bind SARS-CoV2 spike protein S1 subunit and bind to S1 protein and trimerizing S1 protein preventing the binding to ACE-2 receptor of the host cell.

### EXAMPLES

The invention is further illustrated hereinafter by means of examples. The following examples are given by way of illustration only and therefore should not be construed to limit the scope of the present invention.

The sequence listing is provided in computer readable text format along with the specification. The Sequence Listing XML File P1982MY.xml created Aug. 8, 2023 of 8,555 bytes is incorporated herein by reference in its entirety.

The anti-spike monoclonal antibody is from MP biomedicals catalogue number 0720302. S1 spike protein is from Sino Biologicals, Catalogue number 40591-V08H; nitrocellulose membrane is from Axivia Scichem, Delhi, catalogue number: DF20/SX04. The gold nanoparticles are from Sigma, USA, Catalogue number: 741957. Sodium tetraborate decahydrate is from Sigma—catalogue number: S9640-2.5KG. The bovine serum albumin is from Sigma—Catalogue number: A3294-100. The spike S2 protein is from Sino Biologicals, catalogue number: 40590-V08H1. Nickle NTA column is from Bio Rad, USA, catalogue number: 1560123. ACE-2 receptor protein is from Sino Biologicals, Catalogue number: 10108-H05H. The PVDF membrane is from Bio Rad, catalogue number: 88018. ACE-2 is from Sino Bio, Catalogue number: 10108-R003, spike S1 protein using anti-spike antibody is from MP biomedicals catalogue number 0720302 and anti-human beta-tubulin antibody is from Sino Bio, catalogue number: 100109-MM05T.

### Example 1

Designing of the mimic peptides on the structural information available from the crystal structures of SARS-COV spike (Protein data Bank-PDB code 6ACK) and ACE-2 (PDB code 1R42).

The mimic peptides of the present invention were designed based on the structural information available from the crystal structures of SARS-COV spike (Protein data Bank-PDB code 6ACK) and ACE-2 (PDB code 1R42). The structures were analyzed and interaction interfaces were identified using ZDOCK software (http://zdock.umassmed.edu/). The interacting amino acids between spike S1 protein and ACE-2 were identified as listed in FIG. 1 and in the table 2 below.

TABLE 2
The interacting amino acids between human ACE-2
protein and spike S1 protein of SARS-CoV2 virus

| S. | ACE-2 | SARS-CoV-2 spike |
|---|---|---|
| No. | amino acid | protein amino acid |
| 1 | I21, E23, Q24 | R403 |
| 2 | Q24 | G404 |
| 3 | Q24, A25, K26, T27 | D405 |
| 4 | T27 | E406 |
| 5 | F28 | R408 |
| 6 | P28 | Q409 |
| 7 | L29, D30, K31 | K417 |
| 8 | K31 | I418, Y421 |
| 9 | K31, F32 | V445 |
| 10 | N33 | Y449 |
| 11 | N33, H34 | Y453 |
| 12 | H34, E35 | L455 |
| 13 | E35, A36, E37 | F456 |
| 14 | D38 | Y473, Q474, A475, E484 |
| 15 | L39 | G485 |
| 16 | L39, F40, Y41 | F486 |
| 17 | Y41, Q42, S43, S44 | N487 |
| 18 | L45 | C488 |
| 19 | L45, A46, N49, E56, E57 | Y489 |
| 20 | N58, Q60 | F490 |
| 21 | Q60 | L492 |
| 22 | Q60, N61 | Q493 |
| 23 | N61, N64, A65 | S494 |
| 24 | A65, K68 | Y495 |
| 25 | K68 | G496 |
| 26 | K68, F72, E75, Q76 | Q497 |
| 27 | Q76, T78 | P499 |
| 28 | L79, A80 | T500 |
| 29 | A80, Q81, M82, Y83 | N501 |
| 30 | Y38, L100 | G502 |
| 31 | V503 | E329 |
| 32 | G504 | N330, L351, G352 |

The amino acids were further analyzed for the bonding between ACE-2 and SARS-CoV-2 spike. Low energy content, stable structural integrity and interaction were used as way of identifying potential peptide sequences for binding to SARS-CoV-2 or ACE-2. Following 6 peptide sequences were identified and synthesized:

ACE-2 mimics or SARS-CoV-2 spike protein S1 subunit binding peptides:

1. Pep A:
(SEQ ID NO: 1)
YQKDHQM
2. PepB:
(SEQ ID NO: 2)
QKHYQMK
3. PepC:
(SEQ ID NO: 3)
QTFDKNHEDLYLQAKQLK
4. PepD:
(SEQ ID NO: 4)
DKFNHEAEDLFY

SARS-CoV-2 spike protein S1 subunit mimic or ACE-2 binding

5. PepE:
(SEQ ID NO: 5)
QTNKYQFQTNKYQF
6. PepF:
(SEQ ID NO: 6)
KYQFQTNKYQFQTN

Peptides SEQ ID NO: 1 to SEQ ID NO: 4 (PepA to PepD) mimic ACE-2 receptor binding site and have affinity to bind to SARS-CoV-2 spike protein S1 subunit. As shown in the FIG. 1, these peptides SEQ ID NO: 1 to SEQ ID NO: 4, individually or in combinations with other peptides conjugated or modified can bind to SARS-CoV-2 spike S1 protein subunit and stops the virus from entering into cell thus stopping viral entry and infection (antiviral activity).

Peptides SEQ ID NO: 5 and SEQ ID NO: 6 (PepE and PepF) individually or in combinations with other peptides conjugated or modified, mimic the SARS CoV-2 spike protein S1 subunit and bind to S1 proteins while trimerization of S1 protein and also bind to ACE-2 receptor thus competing away the virus from binding and entering the cells for infection (antiviral activity).

### Example 2: Peptide Synthesis Protocol

The peptide synthesis was carried using FMOC (Fluorenylmethyloxycarbonyl protecting group) method where multiple amino acids are linked via amide bonds to form a peptide bond resulting in peptides. This reaction happens by condensation reaction of carboxyl group of one amino acid to amino group of another. Typically, the first amino acid is bound to resin beads functionalized with reactive groups such as amine or hydroxyl group to bind the first amino acid. After first amino acid is bound successive reactions of amino acids are allowed to assemble in successive reactions. Each amino acid is used in the coupling reaction are protected with FMOC.

### Example 3

Interaction of the peptides of SEQ ID NO: 1 to SEQ ID NO: 4 of the present invention, and the virus which binds to ACE-2 receptor on cell surface according to an embodiment of the present invention.

Example 3 and FIG. 1 depict a schematic diagram illustrating how a cell interacting with the virus binds to ACE-2 receptor on cell surface. FIG. 1 illustrates interaction of the peptides of SEQ ID NO: 1, SEQ ID NO: 2, SEQ ID NO: 3, and SEQ ID NO: 4 of the present invention, and the virus which binds to ACE-2 receptor on cell surface according to an embodiment of the present invention. It was observed that in presence of ACE-2 mimicking peptides SEQ ID NO: 1 to SEQ ID NO: 4 (PepA, PepB, PepC, or PepD), individually or in combinations can interact with SARS-CoV-2 S1 protein (mimicking ACE-2 receptor binding domain) and stop from viral infection. The peptides SEQ ID NO: 5 and SEQ ID NO: 6 (PepE and PepF) individually or in combinations can interact with ACE-2 receptor and block virus from interacting with cells or with S1 protein and stop trimerization assembly S1 protein and thus the virus. FIG. 1 showed that the inhibitor mimic peptides (label 3,4) can alone or in combination can mimic the ACE-2 receptor and prevents the binding of virus (label 2) to the said ACE-2 receptor (label 1) thereby preventing the viral infection of the host cell (label 6). The ACE-2 inhibitor peptides of SEQ ID NO: 1 to SEQ ID NO: 4 are designated as peptide A (PepA), peptide B (PepB), peptide C (PepC) and peptide D (PepD) in said FIG. 1. The host cells (label 6) which express Angiotensin Converting Enzyme Receptor-2 (ACE-2) (label 1) usually bind the receptor binding domain (RBD) of spike S1 protein in SARS-CoV2 virus (label 2).

Based on this interaction, a specific approach of identifying the interacting amino acids was devised to develop the peptides of the present invention, which can interact with receptor binding domain (RBD) of spike S1 protein in SARS-CoV2 virus (label 2). The interaction between ACE-2 (label 1) and spike S1 protein was simulated using the structural information available from the crystal structures of SARS-COV spike (Protein data Bank-PDB code 6ACK) and ACE-2 (PDB code 1R42). The structures were analyzed and interaction interfaces were identified using DOCK software (http://zdock.umassmed.edu/). The interacting amino acids between spike S1 protein and ACE-2 were identified as listed above in the table 2 above. The said interacting amino acids were found to be not just linear but were several amino acids were interacting with more than one amino acid suggesting that their interactions were more important. For example, K31 amino acid of ACE-2 interacted with K417, 1418, Y421 and V445 amino acids of spike S1 protein receptor binding domain. Such interactions were identified as important and most required amino acids. Similarly, N33 of ACE-2 receptor interacted with Y449 and Y453 in spike S1 protein, H34 of ACE-2 interacted with Y453 and L455 of spike S1 and E35 interacted with L455 and F456. Using such amino acids and others, several combinations of amino acids resulting in peptides SEQ ID NO: 1, SEQ ID NO: 2, SEQ ID NO: 3, and SEQ ID NO: 4 of the present invention were generated.

### Example 4: Selection of Peptides Based on the Docking Score

Of the several peptides generated by combinations of ACE-2 amino acids interacting with spike S1 protein from the above Table 2, their binding strengths were calculated using docking score as shown in Table 3 below and as shown in FIGS. 2A to 2F.

TABLE 3
Docking score of the interaction between Peptides 1 to 4 and spike S1 protein.

| Rank | 1 | 2 | 3 | 4 | 5 | 6 | 7 | 8 | 9 | 10 |
|---|---|---|---|---|---|---|---|---|---|---|
| Docking Score | −176.931 | −170.254 | −166.682 | −165.844 | −162.202 | −159.616 | −158.795 | −158.400 | −156.506 | −156.289 |
| (Peptide 1) | | | | | | | | | | |
| Docking Score | −178.854 | −175.931 | −175.302 | −172.994 | −171.739 | −171.615 | −171.313 | −170.003 | −168.634 | −168.104 |
| (Peptide 2) | | | | | | | | | | |
| Docking Score | −203.819 | −189.205 | −180.783 | −179.604 | −178.512 | −177.689 | −176.308 | −175.967 | −173.772 | −173.612 |
| (Peptide 3) | | | | | | | | | | |
| Docking Score | −230.184 | −180.790 | −171.114 | −168.702 | −167.229 | −167.070 | −166.150 | −165.109 | −164.346 | −163.865 |
| (Peptide 4) | | | | | | | | | | |
| Docking Score | −213.3 | −211.8 | −208.2 | −206.8 | −204.3 | −203.6 | −203.1 | −201.6 | −199.6 | −196.9 |
| (Peptide 5) | | | | | | | | | | |
| Docking Score | −224.1 | −221.5 | −215.6 | −212.5 | −210.9 | −207.3 | −203.8 | −203.4 | −203 | −201.4 |
| (Peptide 6) | | | | | | | | | | |

From the docking score as shown in FIGS. 2A to 2F and FIGS. 3A-3B, interaction of peptide 3 of SEQ ID NO: 3 was found to be interacting with spike S1 protein (label 8 and 9) showed that there are 10 different binding possibilities with different docking scores (table 1 and 3). Based on the best docking score, this peptide 3 of SEQ ID NO: 3 was selected for further evaluation. The sequence of peptide 3 was identified as (QTFDKNHEDLYLQAKQLK).

Further, from the docking score as shown in FIGS. 2A to 2F and FIGS. 3A and 3C, interaction of peptide 4 of SEQ ID NO: 4 was found to be interacting with spike S1 protein (label 14 and 15) showed that there are 10 different binding possibilities with different 0 docking scores (table 1 and 3). Based on the best docking score, this peptide 4 of SEQ ID NO: 4 was selected for further evaluation. The sequence of peptide 4 was identified as DKFNHEAEDLFY.

### Example 5

Interaction of the peptide of SEQ ID NO: 3 or SEQ ID NO: 4 or peptide complex of SEQ ID NO: 3 and SEQ ID NO: 4 of the present invention, and the virus 5 which binds to ACE-2 receptor on cell surface according to an embodiment of the present invention

As illustrated in FIG. 4A-4C is the interaction of the peptide of SEQ ID NO: 3 or SEQ ID NO: 4 or the peptide complex of SEQ ID NO: 3 and SEQ ID NO: 4 of the present invention, and the virus which binds to ACE-2 receptor on cell surface according to an embodiment of 0 the present invention. It was observed that the mimic peptide of SEQ ID NO: 3 or SEQ ID NO: 4 or the peptide complex of SEQ ID NO: 3 and SEQ ID NO: 4 can mimic the ACE-2 receptor and prevent the binding of virus (label 1) to the said ACE-2 receptor (label 5) thereby preventing the viral infection of the host cell (label 4). The ACE-2 inhibitor mimic peptide of SEQ ID NO: 3 is designated as peptide 3 (PepC) in said FIG. 1 and FIG. 4A. The ACE-2 inhibitor mimic peptide of SEQ ID NO: 4 is designated as peptide 4 (PepD) in said FIG. 1 and FIG. 4B. The host cells (label 4) which express Angiotensin Converting Enzyme Receptor-2 (ACE-2) (label 5) usually bind the receptor binding domain (RBD) of spike S1 protein (label 3) in SARS-CoV2 virus (label 1). Based on this interaction, a specific approach of identifying the interacting amino acids was devised to develop the peptide 3 of SEQ ID NO: 3 or peptide 4 of SEQ ID NO: 4 of the present invention which can interact with receptor binding domain (RBD) of spike S1 protein (label 3) in SARS-CoV2 virus (1). The interaction between ACE-2 (label 5) and spike S1 protein (label 3) was simulated using the structural information available from the crystal structures of SARS-COV spike (Protein data Bank-PDB code 6ACK) and ACE-2 (PDB code 1R42). The structures were analyzed and interaction interfaces were identified using DOCK software (http://zdock.umassmed.edu/). As illustrated in FIGS. 3A-3C, this simulation resulted in 3D model (label 7) of the interaction. Furthermore, the interacting amino acids between spike S1 protein and ACE-2 were identified as listed above in the table 2 that showed the interacting amino acids between human ACE-2 protein and spike S1 protein of SARS-CoV2 virus.

The said interacting amino acids were found to be not just linear but were several amino acids were interacting with more than one amino acid suggesting that their interactions were more important. For example, K31 amino acid of ACE-2 interacted with K417, 1418, Y421, and V445 amino acids of spike S1 protein receptor binding domain. Such interactions were identified as important and most required amino acids. Similarly, N33 of ACE-2 receptor interacted with Y449 and Y453 in spike S1 protein, H34 of ACE-2 interacted with Y453 and L455 of spike S1 and E35 interacted with L455 and F456. Using such amino acids and others, several combinations of amino acids resulting in peptides SEQ ID NO: 1, SEQ ID NO: 2, SEQ ID NO: 3, and SEQ ID NO: 4 of the present invention were generated. As illustrated in FIG. 3B, the interaction of peptide 3 of SEQ ID NO: 3 of the present invention was found to be interacting with spike S1 protein (label 8 and 9). Further, as illustrated in FIG. 3C, the interaction of peptide 4 of SEQ ID NO: 4 of the present invention was found to be interacting with spike S1 protein (label 14 and 15)

### Example 6

Detection of SARS-CoV2 Spike S1 protein with gold nanoparticles conjugated with Peptide 3 (P3) of SEQ ID NO: 3 or Peptide 4 (P4) of SEQ ID NO: 4 of the present invention

In order to test the binding of peptide 3 of SEQ ID NO: 3 or peptide 4 of SEQ ID NO: 4 to spike S1 protein experimentally, 1 g of spike S1 protein (Sino Biologicals, Catalogue number 40591-V08H) was spotted on nitrocellulose membrane (Axivia Scichem, Delhi, catalogue number: DF20/SX04). Peptide 3 of SEQ ID NO: 3 or Peptide P4 of SEQ ID NO: 4 was conjugated to 10 nm-20 nm gold nanoparticles (Sigma, USA, Catalogue number: 741957). The gold nanoparticles and peptide or antibody conjugate were prepared as follows: The gold nanoparticles were centrifuged at 15,000 rpm for 1 hour 45 minutes at 4° C., followed by removal of supernatant and addition of 800 pl of 2 mM sodium tetraborate decahydrate (Sigma—catalogue number: S9640-2.5KG) along with 30 pg of peptide 3 of SEQ ID NO: 3 or peptide of SEQ ID NO: 4 and incubated at 25° C. for 1 hour. After 1 hour, 100 pl of 10% bovine serum albumin (Sigma—Catalogue number: A3294-100) was added and incubated for 1 hour at 25° C. Following the incubation the entire solution was centrifuged at 15,000 rpm for 1 hour 45 minutes at 4° C. The supernatant was removed and 100 pl of 2 mM Sodium tetraborate decahydrate solution was added. Similarly, instead of peptide, anti-spike monoclonal antibody (MP biomedicals catalogue number 0720302) was used to conjugate with gold nanoparticles for positive control.

As illustrated in FIGS. 5 A and 5B, the spike S1 protein spotted on nitrocellulose membrane was incubated with peptide of SEQ ID NO: 3-gold conjugate and antibody-gold conjugate separately. The binding of peptide of SEQ ID NO: 3 (P3)-gold nanoparticles and antibody-gold nanoparticles was observed (label 10) as depicted in FIG. 5A. However, the spike S2 protein (Sino Biologicals, catalogue number: 40590-V08H1) spotted on the same membrane was not detected by the peptide SEQ ID NO: 3 (P3)-gold nanoparticles conjugate (label 10). The anti-spike S1 antibody-gold nanoparticles conjugate was detecting the spike S1 protein (label 11) (FIG. 5A). FIG. 5A showed that peptide SEQ ID NO: 3 (P3) was detecting the spike S1 protein. Furthermore, as illustrated in FIG. 5B, the peptide of SEQ ID NO: 3 (P3)-gold nanoparticles conjugate specifically detected spike S1 protein and not the spike proteins of MERS and SARS-CoV1 viruses (label 12).

As illustrated in FIGS. 6 A and 6B, the spike S1 protein spotted on nitrocellulose membrane was incubated with peptide of SEQ ID NO: 4-gold conjugate and antibody-gold conjugate separately. The binding of peptide of SEQ ID NO: 4 (P4)-gold nanoparticles and antibody-gold nanoparticles was observed (label 16) as depicted in FIG. 6A. However, the spike S2 protein (Sino Biologicals, catalogue number: 40590-V08H1) spotted on the same membrane was not detected by the peptide SEQ ID NO: 4 (P4)-gold nanoparticles conjugate (label 16). The anti-spike S1 antibody-gold nanoparticles conjugate was detecting the spike S1 protein (label 17) (FIG. 6 A). FIG. 6 A showed that peptide SEQ ID NO: 4 (P4) was detecting the spike S1 protein. Furthermore, as illustrated in FIG. 6B, the peptide of SEQ ID NO: 4 (P4)-gold nanoparticles conjugate specifically detected spike S1 protein and not the spike proteins of MERS and SARS-CoVl viruses (label 18).

### Example 7

Experiment with ACE-2 expressing cells HEK293 cells showing proteinprotein interactions of His-Tag recombinant S1 protein and recombinant ACE-2 for binding with and without Peptide-3 of SEQ ID NO: 3 or Peptide-4 of SEQ ID NO: 4 of the present invention

The peptide of SEQ ID NO: 3 (P3) or SEQ ID NO: 4 (P4) to block the interaction between ACE-2 receptor and SARS-CoV2 spike S1 protein was evaluated using a simple method of binding the spike S1 protein to Nickle NTA column. The property of peptide of SEQ ID NO: 3 (P3) or SEQ ID NO: 4 (P4) to Nickle NTA column (Bio Rad, USA, catalogue number: 1560123) and ACE-2 receptor protein (Sino Biologicals, Catalogue number: 10108-H05H) was incubated for 30 minutes and spun down at 5000 rpm for 10 minutes. After removal of supernatant, phosphate buffer saline was added and mixed following by centrifugation at 5000 rpm for 10 minutes. The supernatant was removed and sample loading dye was added for SDS-PAGE (Sodium dodecyl sulphate-polyacrylamide gel electrophoresis). The same reaction was done but along with peptide of SEQ ID NO: 3 or SEQ ID NO: 4 in the reaction tube. The interaction of ACE-2 and spike S1 protein was clearly seen in lane 1 of label 13 of FIG. 7A and was not seen in presence of peptide SEQ ID NO: 3 (P3) (lane 2, label 13 of FIG. 7A). The interaction of ACE-2 and spike S1 protein was clearly seen in lane 2 of label 20 of FIG. 7B and was not seen in presence of peptide SEQ ID NO: 4 (P4) (lane 3, label 20 of FIG. 7C.

As illustrated in FIG. 7A—label 14, an experiment with ACE-2 expressing cells HEK293 cells was carried out. The HEK293 cells were lysed by sonication and these lysed proteins were incubated with Nickle NTA bound spike S1 protein. A similar reaction with and without peptide P3 was carried out and SDS-PAGE was run. This gel for used to transfer the proteins to PVDF membrane (Bio Rad, catalogue number: 88018). This membrane was blotted for ACE-2 using antibody for ACE-2 (Sino Bio, Catalogue number: 10108-R003), spike S1 protein using anti-spike antibody (MP biomedicals catalogue number 0720302) and antihuman beta-tubulin antibody (Sino Bio, catalogue number: 100109-MM05T) as control. The results as depicted in FIG. 7A suggest that the interaction between ACE-2 receptor and spike S1 protein of SARS-CoV2 is blocked in presence of peptide SEQ ID NO: 3 (P3).

As illustrated in FIG. 7C—label 20, an experiment with ACE-2 expressing cells HEK293 cells was carried out. The HEK293 cells were lysed by sonication and these lysed proteins were incubated with Nickle NTA bound spike S1 protein. A similar reaction with and without peptide P4 was carried out and SDS-PAGE was run. This gel for used to transfer the proteins to PVDF membrane (Bio Rad, catalogue number: 88018). This membrane was blotted for ACE-2 using antibody for ACE-2 (Sino Bio, Catalogue number: 10108-R003), spike S1 protein using anti-spike antibody (MP biomedicals catalogue number 0720302) and antihuman beta-tubulin antibody (Sino Bio, catalogue number: 100109-MM05T) as control. The results as depicted in FIG. 7D at label 21 suggest that the interaction between ACE-2 receptor and spike S1 protein of SARS-CoV2 is blocked in presence of peptide SEQ ID NO: 4 (P4).

### Example 8: Protocol for Binding Two Peptides or Proteins to Form a Peptide Complex

As conventionally known, the proteins or peptides in acidic pH tend to unfold and refold to native structure upon making the same mixture to basic pH. In the present experiment, peptide 3 of SEQ ID NO: 3 and peptide 4 of SEQ ID NO: 4 were taken in 2 mM sodium borate at pH 5.8. At this pH the peptides were unfolded and then the mixture pH was slowly adjusted to 8 with 0.1M sodium hydroxide solution. It was observed that the two peptides SEQ ID NO: 3 and SEQ ID NO: 4 in combination tend to come together to form a peptide complex using Vander walls forces of attraction and form a single molecule. This combined peptide complex of SEQ ID NO: 3 and SEQ ID NO: 4 was further used for experimentation to study the combination peptide complex's effect of the ACE-2 receptor binding.

### Example 9

Effect of combination peptides i.e. peptide complex of SEQ ID no: 3 and SEQ ID NO: 4 on the ACE-2 receptor binding to spike S1 protein.

The effect of combination peptide complex of SEQ ID NO: 3 and SEQ ID NO: 4 on the ACE-2 receptor binding was studied. It was observed that in presence of the combination peptide complex of SEQ ID NO: 3 and SEQ ID NO: 4, the ACE-2 receptor binding to spike S1 protein was completely blocked even at 1 ng concentration as seen in FIG. 8.

In accordance with an embodiment of the present invention, in presence of peptide SEQ ID NO: 3 (P3) or peptide SEQ ID NO: 4 (P4) or a peptide complex of SEQ ID NO: 3 and SEQ ID NO: 4 of the present invention, the interaction between human ACE-2 receptor and spike S1 protein is blocked. The process where interaction between two proteins was generated and highly repeated amino acids can be used as backbone for peptide design in accordance to an embodiment of the present invention.

The foregoing description of specific embodiments of the present invention has been presented for purposes of illustration and description. They are not intended to be exhaustive or to limit the present invention to the precise forms disclosed, and obviously many modifications and variations are possible in light of the above teaching.

The embodiments were chosen and described in order to best explain the principles of the present invention and its practical application, to thereby enable others, skilled in the art to best utilize the present invention and various embodiments with various modifications as are suited to the particular use contemplated.

It is understood that various omission and substitutions of equivalents are contemplated as circumstance may suggest or render expedient, but such are intended to cover the application or implementation without departing from the spirit or scope of the present invention.

1. A mimic peptide for specifically binding a SI spike protein of SARS CoV2 virus, the mimic peptide comprising:
an amino acid sequence of SEQ ID NO: 3 or SEQ ID NO: 4.

2. The mimic peptide of claim 1, wherein the mimic peptide is a peptide complex including the amino acid sequence of SEQ ID NO: 3 and SEQ ID NO: 4.

3. The mimic peptide of claim 1, wherein the mimic peptide is conjugated with about 10 nm to 20 nm nanoparticles.

4. The mimic peptide of claim 3, wherein the mimic peptide is a peptide complex including SEQ ID NO: 3 and SEQ ID NO: 4 conjugated with the about 10 nm to 20 nm nanoparticles.

5. The mimic peptide of claim 1 as a treatment composition, further comprising:
a pharmaceutically acceptable carrier.

6. The mimic peptide as a treatment composition of claim 5, wherein the peptide is a peptide complex including SEQ ID NO: 3 and SEQ ID NO: 4 conjugated with about 10 nm to 20 nm nanoparticles.

7. The mimic peptide as a treatment composition of claim 5, wherein the pharmaceutically acceptable carrier includes at least one of, water, saline, phosphate buffered saline, dextrose, glycerol, ethanol, and an isotonic agent including sugar and/or polyalcohol.

8. The mimic peptide as a treatment composition of claim 5, wherein the composition is a nasal spray or a nebulizer-administrated composition.

9. A method of treating or preventing SARS CoV2 infection in a subject, the method comprising:
administering to the subject an effective amount of the mimic peptide of claim 1 so as to inhibit or block the interaction of SARS-CoV2 spike protein with ACE-2 receptor of a host cell of the subject.

10. The method of claim 9, wherein the peptide is a peptide complex including the amino acid sequence of SEQ ID NO: 3 and SEQ ID NO: 4.

11. The method of claim 9, wherein the peptide is conjugated with about 10 nm to 20 nm nanoparticles.

12. The method of claim 11, wherein the peptide is a peptide complex including SEQ ID NO: 3 and SEQ ID NO: 4 conjugated with the about 10 nm to 20 nm nanoparticles.

13. The method of claim 9, wherein the administering is executed through at least one of a nasal spray and a nebulizer.


SEQUENCE LISTING (6 sequences)

SEQ ID NO: 1 - protein, 7 aa, synthetic construct
  REGION 1..7: note = Mimic peptide 1
YQKDHQM

SEQ ID NO: 2 - protein, 7 aa, synthetic construct
  REGION 1..7: note = Mimic peptide 2
QKHYQMK

SEQ ID NO: 3 - protein, 18 aa, synthetic construct
  REGION 1..18: note = Mimic peptide 3
QTFDKNHEDLYLQAKQLK

SEQ ID NO: 4 - protein, 12 aa, synthetic construct
  REGION 1..12: note = Mimic peptide 4
DKFNHEAEDLFY

SEQ ID NO: 5 - protein, 14 aa, synthetic construct
  REGION 1..14: note = Mimic peptide 5
QTNKYQFQTNKYQF

SEQ ID NO: 6 - protein, 14 aa, synthetic construct
  REGION 1..14: note = Mimic peptide 6
KYQFQTNKYQFQTN